(19) United States
(12) Patent Application Publication
(10) Pub. No.: US 2024/0090797 A1
(43) Pub. Date: Mar. 21, 2024

(54) A wearable lumbo-pelvic active balancing device and methods of use

(71) Applicant: Ami Eyal, Modiin lllit (IL)

(72) Inventor: Ami Eyal, Modiin lllit (IL)

(21) Appl. No.: 18/277,224

(22) PCT Filed: Feb. 15, 2022
(86) PCT No.: PCT/IL2022/050175

§ 371 Date: Aug. 15, 2023

(30) Foreign Application Priority Data
Feb. 16, 2021 (IL) 280928

(51) Int. Cl. A61B 5/11; A61B 5/00

(52) U.S. Cl. CPC A61B 5/1116; A61B 5/1121; A61B 5/4566; A61B 5/6831; A61B 2505/09; A61B 2562/02

(57) ABSTRACT

This invention is directed to a wearable device to be worn around a pelvis of an object, comprising: a first compressive element configured to embrace the ASIS and PSIS anatomical structures of the object pelvis; a second compressive element configure to embrace the Ischial Tuberosities and Greater A Trochanter anatomical structures of the object pelvis; at least one sensor for sensing pelvic rotation asymmetry of the object in real time during stride motion; at least two actuators for applying a corrective pressure on at least one PSIS and/or Ischial Tuberosities in real time during said stride motion so as to balance a measured pelvis rotation asymmetry of the object the pelvis rotation; and a control unit configured and operable to receive data in real time from said at least two sensors during the stride motion, calculate the pelvis symmetry based on the data received, and activate at least one of said actuators to apply a corrective pressure upon recognition of a pelvis rotation asymmetry.

### TECHNOLOGICAL FIELD

The invention is related to Lumbo-Pelvic active balancing device in general, and more particular to a novel wearable active training device for neuro-postural pelvic balancing.

### BACKGROUND

Low back pain (LBP) is a common health problem worldwide and a major cause of disability affecting performance at work and general well-being. Low back pain can be acute, sub-acute, or chronic. Low back pain affects people of all ages, from children to the elderly, and is a very frequent reason for medical consultations. In the USA, back pain accounts for more than 264 million lost working days in a year. Approximately 70-80% of people experience symptoms of LBP at least once in their lifetime, and approximately one quarter of adults in the United States report experiencing LBP in the past 3 months:
https://www.optumlabs.com/spotlights/optumlabs-spotlights/back-pain-opportunities.html; https://www.ncbi.nlm.nih.gov/pmc/articles/PMC6882374/; https://jamanetwork.com/journals/jama/article-abstract/2616379).

Such statistics show the clear need to improve quality of life for people with LBP through treatments that adhere to clinical guidelines, which recommend first-line treatments of exercise, time and non-invasive therapies like physical therapy and chiropractic care and discourage the use of opioids and unnecessary complex surgeries.

Recent research conducted by OptumLabs and Boston University School of Public Health provides insight into opportunities for better care options (https://www.optumlabs.com/spotlights/optumlabs-spotlights/back-pain-opportunities.htm). The research finds that a patient's initial choice in health care provider (i.e., seeing a non-invasive therapist first, vs. a primary care physician for a new episode of LBP), results in much lower odds of early and long-term opioid use. More than half of regular opioid users report back pain. Complications of opioid use include addiction and overdose related mortality, which have risen in parallel with prescription rates, leading to the opioid crisis: 46 people per day, or almost 17,000 people per year, die from overdoses of chronic pain killing opioid drugs. Other study published at the Journal of the American Medical Association found that too many complex back surgeries are being done and patients are suffering as a result from complications. The study also show that most back surgeries make a minimal difference. The findings also suggest that health plans could play an important role in making it easier for consumers to consider non-invasive therapies for LBP.

LBP has long been connected to postural and structural Asymmetries, most commonly in the pelvis. Pelvic asymmetry refers to asymmetric pelvic alignment, with respect to the vertical axis, in the frontal or sagittal planes. Pelvic asymmetry in the sagittal plane, namely, iliac rotation asymmetry, is often linked to sacroiliac joint dysfunction, and refers to malalignment between the left and right innominate bones. This malalignment could either be unilateral anterior or posterior rotation of one innominate bone or bilateral contra-movement of the innominate bones when both innominate rotate in opposite directions. In the frontal plane, failure of the pelvis to lie in a perfectly horizontal position is commonly called lateral pelvic tilt. It is presumed that pelvic asymmetry alters the body mechanics, puts various body segments under strain, and, therefore, contributes to musculoskeletal pain. Compensation for pelvic asymmetry that occurs in the musculoskeletal system alters the mechanics of the lumbar spine as reflected, for example, by altered movement patterns in a standing position. A more detailed description of pelvic asymmetry implications can be found In Einas Al—Eisa et al., “Effects of Pelvic Asymmetry and Low Back Pain on Trunk Kinematics During Sitting: A Comparison With Standing” (SPINE Volume 31, Number 5, pp E135—E143, 2006).

Thus, there is a need for another solution for LBP therapy, that is non-invasive and safe. There is a need for automated device that will provide fast, efficient, and individualized assessment and treatment for the mechanical root cause of LBP. A self-regulated advantage, addressing long term treatment goals and prevention from degenerative deterioration process.

The present invention is directed to a novel wearable active training device for neuro-postural pelvic balancing that allows for reduction and prevention of musculoskeletal pain related to the back area and extremities, mainly by sensing and correcting bilateral pelvic rotation angle, pelvic asymmetry and spinal center of gravity, as will be described in detail hereinbelow.

### SUMMARY OF THE INVENTION

In one main aspect, this invention is aimed to provide a Lumbo-Pelvic Active Correction Trainer device and system (denoted herein after: “LPACT device” and

“LPACT system” respectively). The terms ‘IPACT device’, ‘wearable LPACT device’, ‘lumbo Pelvic device’, ‘wearable Lumbo Pelvic device’, ‘Lumbo Pelvic wearable device’, ‘wearable device’, ‘wearable active training device’ and ‘Lumbo Pelvic active balancing device’ may be used interchangeably and are all meaning the same.

The LPACT device is a wearable device directed to active reduction and prevention of back pain that is based on sensing bilateral pelvic angle of an object, comparing the right and left pelvic rotation angle of both sides and correcting pelvic asymmetry and spinal center of gravity differentially according to the data measured in real time during walking. Correction is made by actuating counteractive pressures to the posterior pelvis portion in specific anatomical locations during a short walk, based on the measured data.

The LPACT device of the invention may also be used to reduce and prevent other musculoskeletal pain related to the back and extremities.

In some embodiments of the invention, a wearable device for active neuro mechanical training configured to be worn around a pelvis of an object, is provided. The wearable device comprising: a) a first compressive element configured to embrace the ASIS and PSIS anatomical structures of the object pelvis and to be fastened on these anatomical structures by at least one fastening element; b) a second compressive element configure to embrace the Ischial Tuberosities and Greater Trochanter anatomical structures of the object pelvis and to be fastened on these anatomical structures by at least one fastening element; c) at least one sensor for sensing pelvic rotation asymmetry of the object in real time during stride motion; d) at least two extendable actuators for applying a corrective pressure on at least one PSIS and/or Ischial Tuberosities in real time during said stride motion, so as to balance a measured pelvis rotation asymmetry of the object the pelvis rotation; e) at least two size adjusting elements to allow positioning of said sensors and actuators onto the anatomical structures according to the specific dimensions of the object; f) at least two fastening elements for securing said first and second compressive elements onto the anatomical structures of the object pelvis; and g) a control unit. The control unit is configured and operable to receive data in real time from the at least one sensor during the stride motion, calculate the pelvis symmetry based on the data received, and activate at least one of said extendable actuators to apply a corrective pressure upon recognition of a pelvis rotation asymmetry.

In one optional embodiment, the sensor is at least two sensors positioned on the first compressive element onto the right and left ASIS anatomical elements and configured to provide data about the pelvic rotation angle during right gait and left gait, or at least one sensor configured to be positioned on the vertebrates of the object and to provide data indicative of Pelvis motion along X, Y, Z axis during stride motion, or combination thereof. For the purpose of this invention, the sensors may be either one of the following: stretch sensors, motion sensors, pressure sensors, location sensors, electronic skin sensors, conductive sensors, and combination thereof.

In some embodiments of the invention, the at least two actuators are positioned in a manner that at least one actuator is placed on the first compressive element onto the right and/or left PSIS anatomical elements, and at least one another actuator is placed on the second compressive element onto the right and/or left Ischial Tuberosities.

In some other embodiments of the invention, the wearable device comprises four actuators, two positioned on the first compressive element, one on the right PSIS and the other on the left PSIS, and two positioned on the second compressive element, one on the right Ischial Tuberosities and the other on the left Ischial Tuberosities.

Preferably, the actuators and sensors are continuously sending data to the control unit during stride motion and the correction of the pelvis rotation asymmetry detected is performed in real time until the right and left rotation angles of the pelvis are similar. The similarity of the rotation angle between the right and the left sides is indicative of a balanced pelvis. In a preferred embodiment, the stride motion and pelvis asymmetry detection and correction are performed in periodic sessions of up to ten minutes daily, weekly or monthly according to the pelvis rotation asymmetry detected.

In some embodiments of the invention, the actuators are volume-controlled elements and the pressure imposed to balance the pelvis asymmetry is correlative to the volume of the actuator in use.

In some none limiting examples, the extendable actuators are either one of the following elements: a) inflatable elements configured to be inflated with gas; b) electroactive elements (EAP's) configured to change volume upon changing physical state, for example, by inducing electric current; and c) fabric soft pneumatic actuators (FSPAs).

In accordance with embodiments of the invention, the electroactive elements may be made of electroactive polymers, such as but not limited to, dielectric elastomers, electro-responsive polymers, Ionic electroactive polymers (IEAP's) and conductive polymers.

In some optional embodiments, the actuators are capable to change volume in a differential manner such that the pressure applied in one area of the actuator is different than the pressure applied in another area of the actuator. This embodiment may be applicable when using one actuator that covers more than one anatomical structure. In such case, the balancing pressure/force applies is directed only to the anatomical spot that needed for balancing the pelvis asymmetry. As mentioned above, in some other optional embodiments, the wearable device comprises at least four actuators, each actuator is positioned onto specific anatomical structure.

The first compressive element and the second compressive element may be housed together in a form of a belt, a garment, and a pouch. The wearable device may further comprise a screen. The screen may be for presentation of data or a touch screen that can also be used to operate the wearable device.

In some embodiments, the at least two size adjusting elements configured to allow positioning of the sensors and actuators onto the anatomical structures described above, are at least one slider connected to a sliding strip to thereby allow sliding of at least one of the compressive elements upward and downward on the sliding strip for positioning said actuators and sensors on said anatomical structures of the pelvis according to the specific dimensions of the object.

In some optional embodiment, the wearable device may further comprise wireless communication elements to allow connection of the wearable device to a smart device that comprises a supportive software or App for communicating with the wearable device. The analysis of the data received from the sensors, determining pelvis asymmetry and calculation of the corrective force to be applied on the object pelvis to reach symmetry may be calculated in real time by the control unit or by the smart device or by a remote computer.

The present invention is also directed to a Lumbo Pelvic active balancing device for reduction and prevention of back pain, said balancing device is a wearable device configured to allow sensing bilateral pelvic angle of an object, comparing the right and left pelvic rotation angles and correcting a pelvic asymmetry according to the data measured in real time during stride motion.

In some embodiments of the invention, the Lumbo Pelvic active balancing device comprises a first compressive element configured to embrace the ASIS and PSIS anatomical structures of the object pelvis; a second compressive element configure to embrace the Ischial Tuberosities and Greater Trochanter anatomical structures of the object pelvis; at least one sensor for sensing pelvic rotation asymmetry of the object in real time during the stride motion; at least two extendable actuators for applying a corrective pressure on at least one PSIS and/or Ischial Tuberosities in real time during said stride motion so as to balance a measured pelvis rotation asymmetry of the object; at least two size adjusting elements to allow positioning of said sensors and actuators onto the said anatomical structures according to the specific dimensions of the object; and a control unit.

In some optional embodiments, the sensor are at least two sensors positioned on the first compressive element onto the right and left ASIS anatomical elements and configured to provide data about the pelvic rotation angle during right gait and left gait, or at least one sensor configured to be positioned on the vertebrates of the object and to provide data indicative of Pelvis motion along X, Y, Z axis during stride motion, or combination thereof. The at least two extendable actuators may be positioned in a manner that at least one actuator is placed on the first compressive element onto the right and/or left PSIS anatomical elements, and at least one another actuator is placed on the second compressive element onto the right and/or left Ischial Tuberosities.

In a preferred embodiment, the extendable actuators and sensors are continuously sending data to the control unit during stride motion and the correction of the pelvis rotation asymmetry detected is performed in real time until the right and left rotation angles of the pelvis are similar.

The stride motion and pelvis asymmetry detection and correction may be performed in periodic sessions of up to ten minutes daily, weekly or monthly according to the pelvis rotation asymmetry detected.

In some embodiments, the extendable actuators are volume-controlled elements and the pressure imposed to balance the pelvis asymmetry is correlative to the volume of the actuator in use. The extendable actuators may be either one of the following elements: a) inflatable elements configured to be inflated with gas; b) electroactive elements (EAP's) configured to change volume upon changing physical state; and c) a fabric soft pneumatic actuator (FSPA).

In some optional embodiments, the actuators are capable to change volume in a differential manner such that the pressure applied in one area of the actuator is different than the pressure applied in another area of the actuator. This embodiment may be applicable when using one actuator that covers more than one anatomical structure. In such case, the balancing pressure/force applies is directed only to the anatomical spot that needed for balancing the pelvis asymmetry.

As mentioned above, in some other optional embodiments, the Lumbo Pelvic active balancing device comprises at least four actuators, each actuator is positioned onto specific anatomical structure.

Optionally, the first compressive element and the second compressive element are housed together in a form, for example but not limited to a belt, a garment, and a pouch.

The Lumbo Pelvic active balancing device may comprise a screen or a touch screen and may further comprise at least two size adjusting element to allow positioning of the sensors and actuators onto the anatomical structures described above. Optionally, the size adjusting elements are at least one slider connected to a sliding strip to thereby allow sliding of at least one of the compressive elements upward and downward on the sliding strip for positioning said actuators and sensors on said anatomical structures of the pelvis according to the specific dimensions of the object.

In some optional embodiments, the Lumbo Pelvic active balancing device is further comprising a wireless communication element so as to allow connection of the device to a smart device. The smart device may comprise a supportive software or App for communicating with the device. The analysis of the data received from the sensors, determining pelvis asymmetry and calculation of the corrective force to be applied on the object pelvis to reach symmetry may be calculated in real time by the control unit or by the smart device. Alternatively, it can be made by a remote computer.

As mentioned above, the operation of the device is configured for short time periods of few minutes, preferably, at least once a day within a training term. The mechanical function of this usage during the training term on the object's body leads to an active mechanoreceptive neuro-muscular training for low back and lumbopelvic posture balancing. The training process is aimed to create a long-lasting postural balance change for reducing lumbar-intervertebral discs, lumbopelvic, sacroiliac and hip joints Asymmetrical pressure loads, in order to prevent/reduce degenerative damages to these areas and other spinal segments and lower extremities joints.

In some further aspects, the invention is directed to a method for active neuro mechanical training of an object, the method comprising at least the following steps: a) positioning a wearable device according to the above around a pelvis of an object, such that a first compressive element is configured to embrace the ASIS and PSIS anatomical structures of the object pelvis, and to be fastened on these anatomical structures by at least one fastening element; and a second compressive element is configure to embrace the Ischial Tuberosities and Greater Trochanter anatomical structures of the object pelvis and to be fastened on these anatomical structures by at least one fastening element; b) adjusting the size of the wearable device by at least two size adjusting elements to allow positioning of said sensors and actuators onto the anatomical structures according to the specific dimensions of the object; c) fastening the compressive elements by at least two fastening elements for securing said first and second compressive elements onto the anatomical structures of the object pelvis; d) sensing the pelvic rotation asymmetry of the object in real time during stride motion by at least one sensor; and e) applying a corrective pressure on at least one PSIS and/or Ischial Tuberosities in real time during said stride motion by at least two expandable actuators, to balance a measured pelvis rotation asymmetry of the object; wherein, a control unit is configured and operable to receive data in real time from said at least one sensor during the stride motion, calculate the pelvis symmetry based on the data received, and activate at least one of said expandable actuators to apply an adaptive corrective pressure upon recognition of a pelvis rotation asymmetry. In some optional implementation of the he method for active neuro mechanical training provided herein, the sensor is at least two sensors positioned on the first compressive element onto the right and left ASIS anatomical elements and configured to provide data about the pelvic rotation angle during right gait and left gait, or at least one sensor configured to be positioned on the vertebrates of the object and to provide data indicative of Pelvis motion along X, Y, Z axis during stride motion, or combination thereof. The sensor may be selected from the group consisting of: stretch sensors, motion sensors, pressure sensors, location sensors, electronic skin sensors, and conductive sensors. Additionally, the at least two expandable actuators may be positioned in a manner that at least one actuator is placed on the first compressive element onto the right and/or left PSIS anatomical elements, and at least one another actuator is placed on the second compressive element onto the right and/or left Ischial Tuberosities. The sensors are continuously sending data to the control unit during stride motion and the correction of the pelvis rotation asymmetry detected is performed in real time by adjusting the corrective pressure level applied by said actuators until the right and left rotation angles of the pelvis are similar. Preferably, the stride motion and pelvis asymmetry detection and correction are performed in periodic training sessions of up to ten minutes daily, weekly or monthly according to the pelvis rotation asymmetry detected.

### BRIEF DESCRIPTION OF THE DRAWINGS

Examples illustrative of embodiments of the disclosure are described below with reference to figures attached hereto. Dimensions of components and features shown in the figures are generally chosen for convenience and clarity of presentation and are not necessarily shown to scale. Many of the figures presented are in the form of schematic illustrations and, as such, certain elements may be drawn greatly simplified or not-to-scale, for illustrative clarity. The figures are not intended to be production drawings.

### The Figures (FIGS.) are Listed Below.

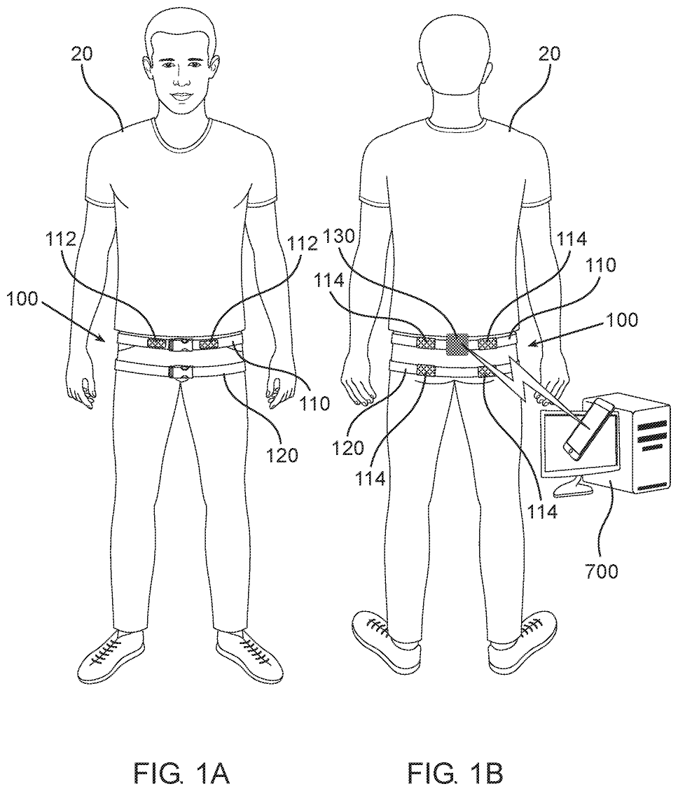

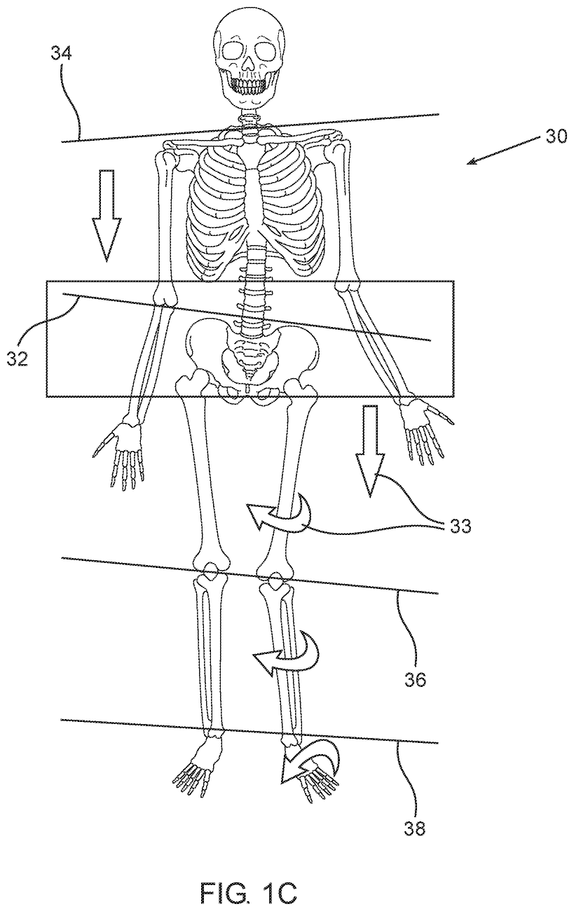

FIGS. 1A and 1B are schematic front view and back view illustrations of one example of LPACT device and system comprising an upper compressive belt and a lower compressive belt according to one possible embodiment of the invention.

FIG. 1C is a schematic illustration demonstrating how compensations for pelvic asymmetry which may occurs in the musculoskeletal system alters the skeleton mechanics, puts various body segments under strain, and contributes to musculoskeletal disfunction and pain in other areas of the body.

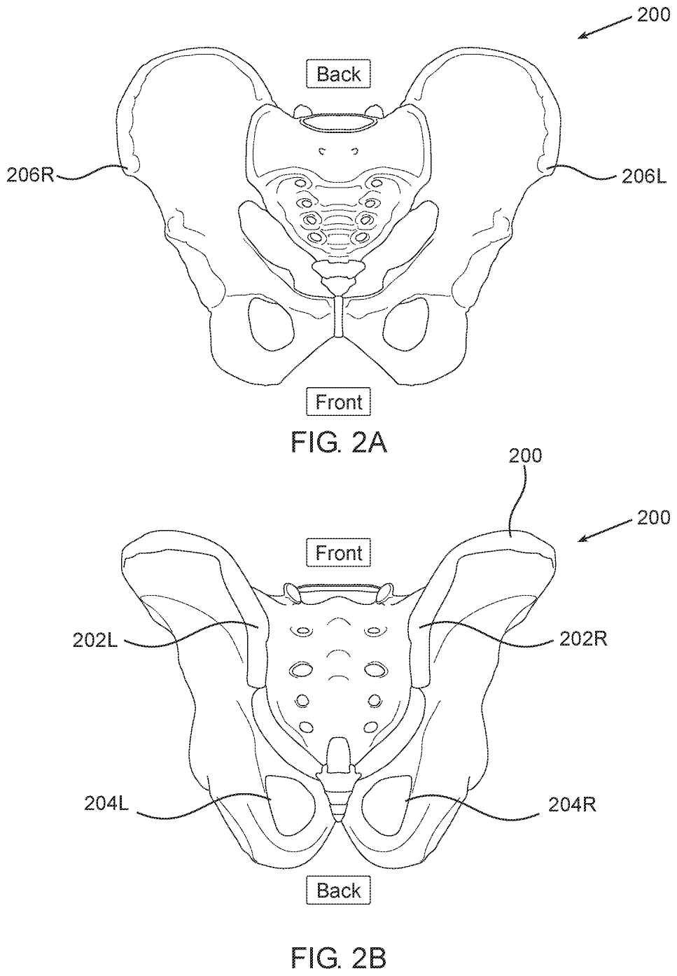

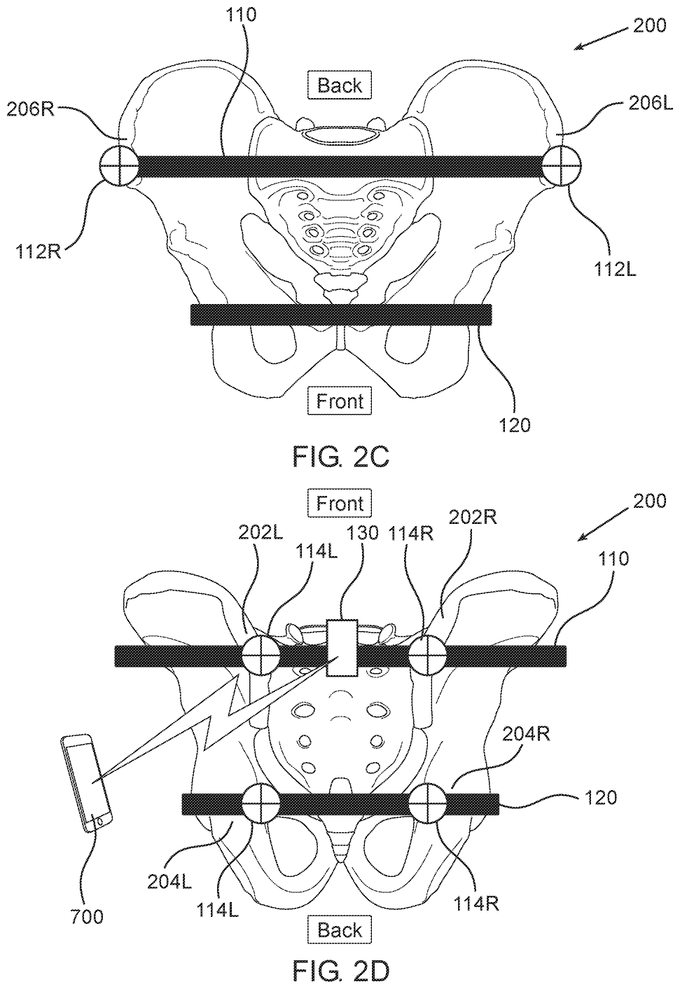

FIGS. 2A-2B are schematic back and front view illustrations showing the main anatomical structures of human pelvis from anterior and posterior views respectively.

FIGS. 2C-2D are schematic back and front view illustrations of the human pelvis 200 and components of the LPACT device 100 as positioned on the anatomical structures of the pelvis.

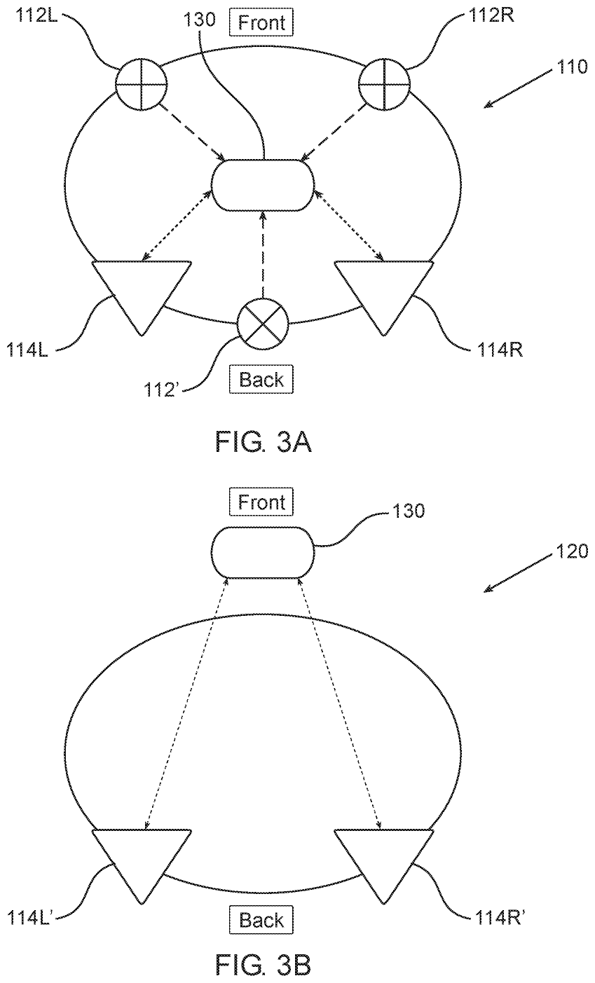

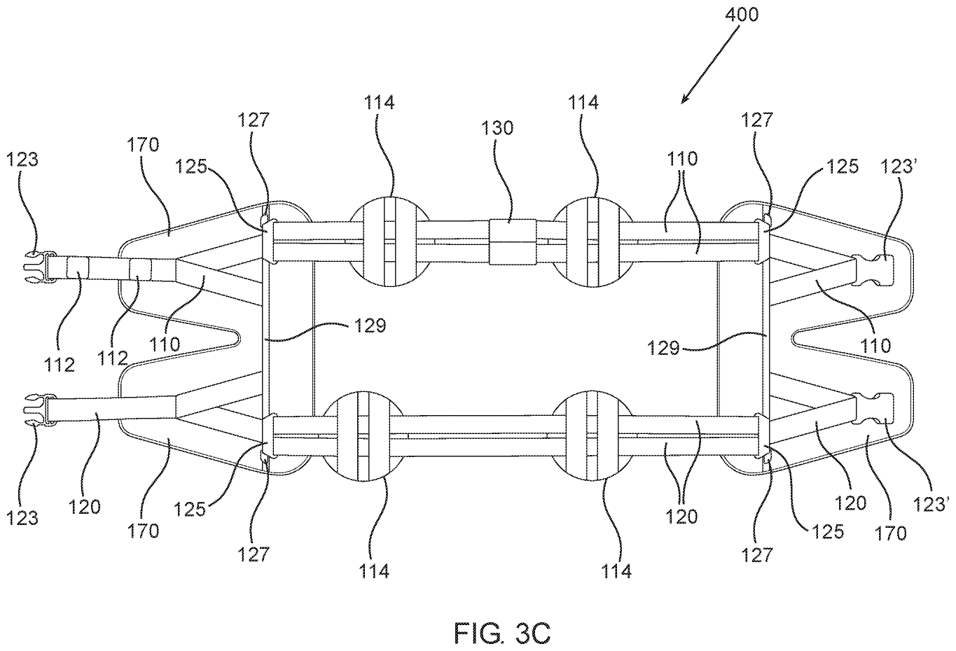

FIGS. 3A-3B are schematic block diagram illustrations showing the main components comprised in the upper compressive element (3A) and the lower compressive element (3B) of the LPACT device of FIG. 1A according to one optional embodiment of the invention.

FIG. 3C is a schematic illustration of one optional example of the LPACT wearable device of the invention shaped as a belt.

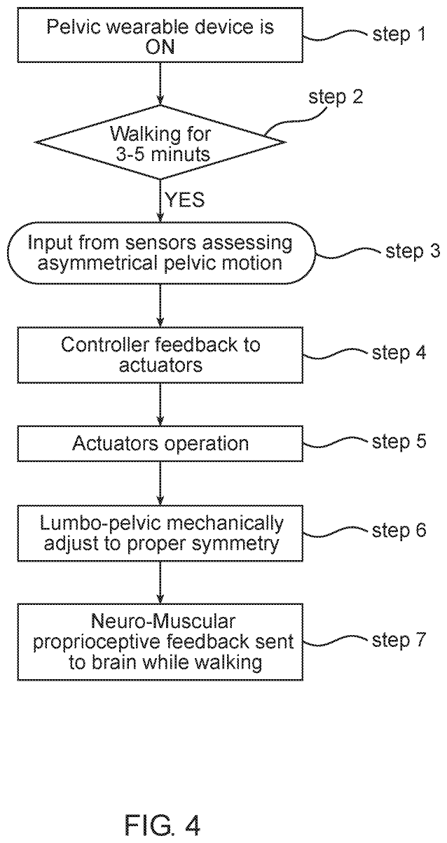

FIG. 4 is a flow chart diagram illustrating the main functional operative steps of the LPACT device of FIG. 1 according to one optional embodiment of the invention.

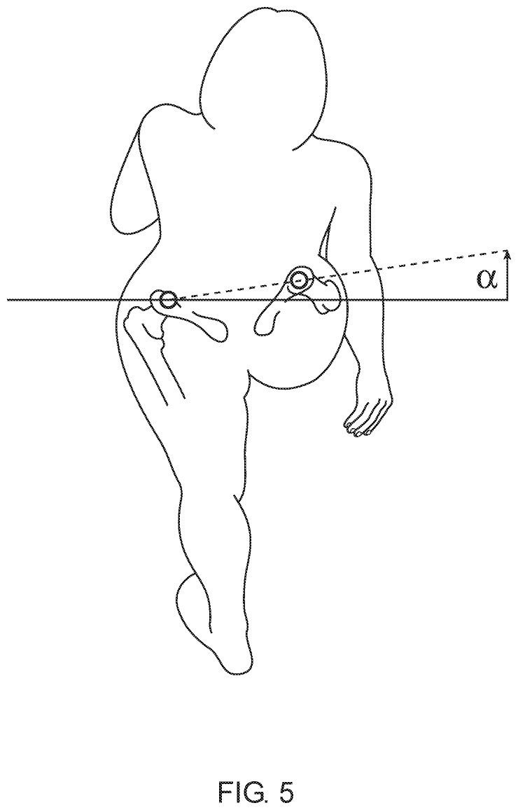

FIG. 5 is a schematic back view illustration of a walking object showing the pelvic rotation angle created on during right gait.

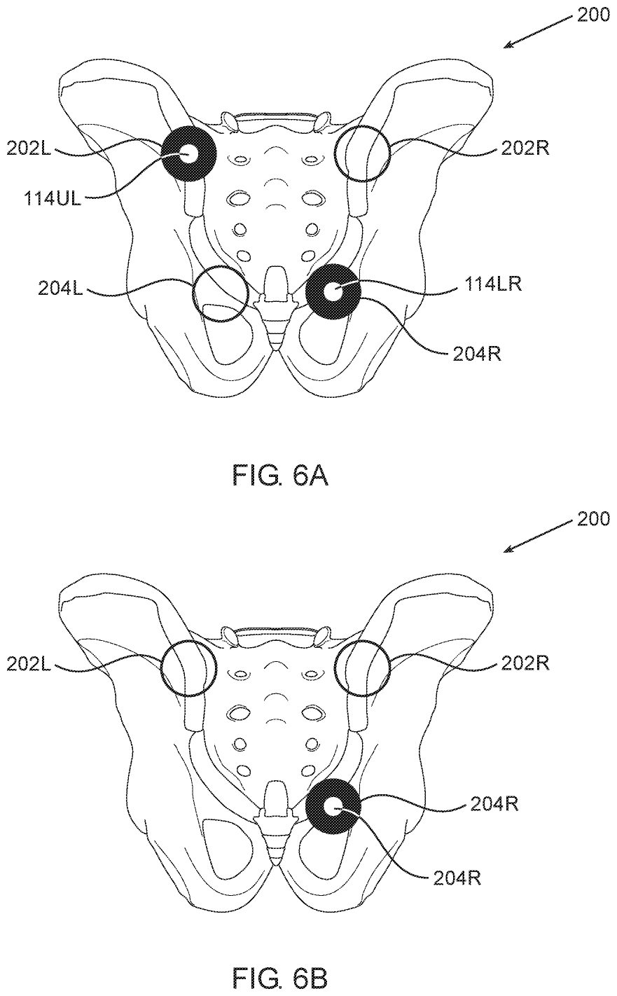

FIGS. 6A-6B are schematic illustrations of actuating balancing forces in two optional configurations of Lumbo-Pelvic failure (FIG. 6A) and Pelvic failure (FIG. 6B).

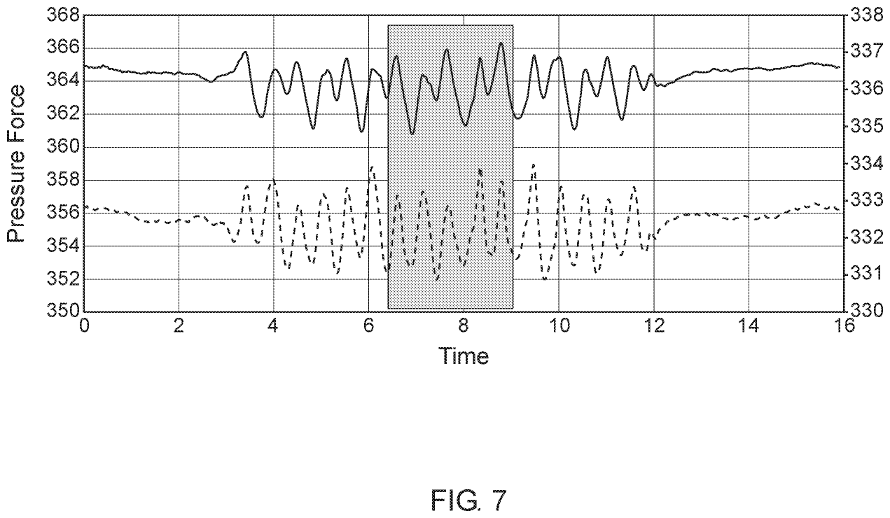

FIG. 7 is one example of data view graph of a LPACT system according to embodiments of the invention.

### DETAILED DESCRIPTION OF EMBODIMENTS

In the following description, various aspects of LPACT device and system will be described. For the purpose of explanation, specific configurations and details are set forth in order to provide a thorough understanding of the invention.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment. Furthermore, it should be understood that the disclosure can be carried out or practiced in various ways, and that the disclosure can be implemented in embodiments other than the exemplary ones described herein below. The descriptions, examples and materials presented in the description, as well as in the claims, should not be construed as limiting, but rather as illustrative.

The present invention provides a novel wearable active training device for neuro-postural pelvic balancing that allows for reduction and prevention of musculoskeletal pain related to the back area and extremities.

In a preferred embodiment, the wearable device comprises two main compressive elements shaped like a compressive belt, an upper belt and a lower belt positioned around the pelvic and connected one to the other structurally and electronically. Assembly of the device on the object's body is performed by embracing the device on the object pelvis and positioning of some components onto specific anatomic structures elements of the pelvis. The upper compressive belt component is interfacing in the front side to the anterior superior iliac spine (ASIS) on both sides of the pelvis and the back side is aligned with the posterior superior iliac spine (PSIS) on both sides. The lower compressive belt component is aligned with the two Ischial Tuberosities structures and the hip joints on the sides of the pelvic.

Reference is now made to the figures.

FIGS. 1A-1B are schematic front view and back view illustration of a wearable LPACT device 100. In one none-limiting embodiment, the LPACT device comprises two compressive elements, a superior (upper) compressive element 110 and an inferior (lower) compressive element 120 having electrical communication therebetween. The two compressive elements may be housed together such that the object will wear one belt like device comprising both compressive elements. Alternatively, the two compressive elements may be implemented as garment or accessory. For example, as a tights, pants, top, pouch, or belt. In the specific example illustrated in these figures the compressive elements are designed as compressive belts. The belts are functionally connected to each other by a control unit 130 and allow to detect posture asymmetry and regaining pelvis rotation balance. The connection between the two compressive belts may be in terms of electronic wiring and/or wireless connection, common housing, and operational competence.

The LPACT device can be connected to at least one smart device 700, such as but not limited to a computer, a tablet, a smart phone, a smart watch, or a smart bracelet having a dedicated App or software, thereby creating the LPACT system. In some embodiments the LPACT system is also connected to cloud-based database and/or to therapist's center or a physician that monitor the data and support the user accordingly.

In order to achieve optimal posture balance, the LPACT device wearing is specifically fixed to pelvic anatomical structures as will be descried in detail with reference to FIGS. 2A-2B hereinbelow.

The upper compressive element 110 may comprise at least one sensor 112. In some optional embodiment upper compressive element 110 comprises two sensors positioned at the anterior (front) side of the pelvis as shown in FIG. 1A. In some other optional embodiments, upper compressive element 110 comprises at least one sensor positioned at the back side of the body. In one another optional embodiment, the upper belt may comprise two front sensors and one back sensor (illustrated in FIG. 3A). Yet, in some other optional embodiment upper compressive element 110 may comprise an array of sensors. The upper compressive element 110 further comprises at least two actuators 114 that are preferably positioned at the posterior (back) side of the object (FIG. 1B).

Various sensors may be used to measure the pelvic rotation angle. Some optional none limiting examples are pressure sensors, stretch sensors (like Dielectric Elastomers and E. Skin sensors), and position sensors (like Accelerometers, Gyroscopes and magnetometers). Any other sensor that allows calculation of the pelvic rotation angle during walking may also be used and considered within the scope of this invention.

The sensors are functionally capable of measuring/determining pelvis posture by providing indication about the pelvic rotation angle at the right side and the left side of the pelvic during walking. The data obtained is transmitted to a control unit 130 as will be described in detail with reference to FIGS. 4 and 5 hereinafter.

Lower compressive element 120 comprises at least two actuators 114 positioned at the posterior (back) side of the object. Actuators 114 of both upper and lower belts are preferably but not necessarily, mechanical elements such as but not limited to Dielectric elastomers, Inflatable bladders and vibrating motors. These mechanical elements are configured to apply pressure/force and/or to stimulate specific posterior anatomical locations of the pelvis, such as the posterior superior Iliac spines, Sacro-iliac joints, Lumbo-Pelvic (L5-S1) joint, and the Ischial tuberosities. Optionally, the stimulating means may be embedded within the LPACT device. Alternatively, the stimulating means can be a separated off—the—shelf articles that are used for stimulation.

FIG. 1C is a general schematic illustration of a skeleton 30 demonstrating how compensations for pelvic asymmetry that may occurs in the musculoskeletal system alters the skeleton mechanics, puts various body segments under strain, that can contribute to musculoskeletal disfunction and pain in other areas besides the lower back. It is presumed in the art that pelvic asymmetry alters the body mechanics, puts various body segments under strain, and, therefore, contributes to musculoskeletal pain.

This figure illustrates how compensation for pelvic asymmetry that occurs in the musculoskeletal system alters the skeleton 30 mechanics, puts various body segments under strain, and contributes possibly, to musculoskeletal disfunction and pain in the lower back and in other areas as well. For example, asymmetrical oblique rotated Pelvis 32 can increase pressure load on the left leg and internal hip. It can further result in left knee and leg internal rotation 36, internal rotation of the left ankle and foot 38 and uneven shoulders, with dropping of the right shoulder 34. It should be clear that the musculoskeletal disfunctions mentioned above with reference to FIG. 1C are only some none-limiting examples and not all possible musculoskeletal disfunctions that may occur as a result of asymmetrical rotated pelvis. A more detailed description of pelvic asymmetry implications can be found in the following reference: Einas Al-Eisa et al., “Effects of Pelvic Asymmetry and Low Back Pain on Trunk Kinematics During Sitting: A Comparison with Standing” (SPINE Volume 31, Number 5, pp E135—E143, 2006.

FIGS. 2A-2B are schematic back and front view illustrations showing the main anatomical structures of human pelvis 200 from anterior and posterior views respectively, and FIGS. 2C-2D are schematic back and front view illustrations of the human pelvis 200 and components of the LPACT device 100 as positioned on the pelvis anatomical structures.

Upper compressive belt 110 is configured to be positioned at its front portion on the Anterior Superior Iliac Spines (ASIS) 206 pelvic anatomical structures on both sides (right and left), while its back portion is positioned on the Posterior Superior Iliac Spines (PSIS) 202 pelvic anatomical structures on both sides.

Lower compressive belt 120 is configured to be positioned at its back portion to the Ischial Tuberosities 204 pelvic anatomical structures on both sides and to the Greater trochanters hips anatomical structures (not shown) at both sides. As can be seen in the front view (FIG. 2A) sensors 112 are positioned at the front on right and left ASIS 206, while in the back view (FIG. 2B) upper actuators 114 are positioned on PSIS 202 on both sides, and the lower actuators 114 are positioned on the Ischial Tuberosities 204 pelvic anatomical structures on both sides. Since the actuators are overlapping the anatomical structures, for simplicity of description, the lines that are pointing to 204L and 204R are placed adjacent to these structures.

As the positioning of the wearable LPACT device is essential to the measured parameters, the position of the compression elements is adjustable even when the two compressive elements are assembled in a single housing, in a manner that the (height) of each compressive element within the housing is tunable to allow positioning of the actuators and sensor on the proper anatomical structures as described above. Additionally, the length of the compressive elements can be adjusted and set according to the pelvis perimeter of the object. It should be clear that although the description above refers to two compressive elements, this invention is not limited to this number of compressive elements and this invention may be implemented by using a single width compressive element, or a plurality of thin compressive elements to accomplish the same. In a similar manner, the number of sensors and actuators may vary, and the above example is just for simplicity of description and not intended to limit the scope of this invention in any manner.

FIGS. 3A-3B are schematic block diagram illustrations showing the main components comprised in the upper compressive element (FIG. 3A) and the lower compressive element (FIG. 3B) of LPACT device 100 according to one optional embodiment of the invention for simplicity of description, both compressive elements are shaped as single compressive belts.

Upper compressive belt 110 comprises at the back side a right upper actuator 114 located on the right PSIS 202, and a left upper actuator 114 located on the left PSIS 202L, and a control unit 130. At the front side upper compressive belt 110 comprises right sensor 112 located on the right ASIS and left sensor 112 located on the left ASIS. In a preferred embodiment sensor 112 is a stretch sensor. Optionally, upper compressive belt 110 may comprise additional sensor 112′ preferably positioned at the center of the back portion of the belt. In such embodiment sensor 112′ may be a motion sensor and preferably located on the spine at L5-S 1 vertebrate level.

The sensors are functionally connected to control unit 130 that received the data during walking and activate actuators 114 according to the measured data in real time. Detailed description of the activity flow will be described below with reference to FIGS. 4-5. Control unit 130 preferably comprises at least a controller and electric components to receive the data obtained from

Lower compressive belt 120 comprises at the back side a right lower actuator 114R′ located on the right Ischial Tuberosity 204R, and a left lower actuator 114L′ located on the left Ischial Tuberosity 204L. The two lower actuators are functionally connected to controller 130 of upper belt 110 and upon the activated according to the controller's commands during walking.

FIG. 3C is a schematic top view illustration of one optional example of the LPACT wearable device of the invention shaped as a belt.

In the specific example illustrated in this drawing first compressive element 110 and second compressive elements 120 are housed together to form a belt 400. The two compressive elements are connected at the right and left edges to fabric 170, each compressive element is connected to fabric 170 through a slider 125 assembled onto a sliding strip 129 that allows sliding the compressive element upward and downward for positioning of the actuators 114 on the proper anatomical structure of the pelvis according to the specific dimensions of the object. Each end of sliding strip 129 comprises a stopper 127 for securing the sliders 125 from being detached from sliding strip 129. Each one of the compressive belts comprises a buckle. In the specific example illustrated herein the buckle is composed of two components, first component 123 that is configured to be inserted into second component 123′ to close the compressive belt around the object pelvis. It should be clear that any other type of buckle can be used, and the buckle illustrated in this figure is a none limiting example. Also shown in this figure are sensors 112 and control unit 130.

FIG. 4 is a flow chart diagram illustrating the main functional operative steps of the LPACT device of FIG. 1 according to one optional embodiment of the invention. In step one (1) LPACT wearable device 100 is being worn on an object's pelvis an turned “ON”. In step two (2) the object starts walking and/or running (for 3-5 minutes). At step three (3) data from the sensors is transmitted to the control unit and the data is assessed to indicate symmetrical/asymmetrical pelvic motion. In step four (4) if asymmetry of the pelvis motion was recognized, the control unit provides instructions and activate the relevant actuators to apply pressure on posterior pelvis key points. At step five (5) the actuators are acting according to the activation instructions received from the control unit and apply pre-determined pressure on the object according to real time data obtained from the sensors during walking and feedback is sent back to the control unit. At step six (6) the Lumbo-Pelvic failure/Pelvic failure detected is mechanically being adjusted to proper symmetry following the triggers obtained from the actuators (by the counter pressure applied). The six main Lumbo-Pelvic and Pelvic failures that can be balanced by the LPACT wearable device of the invention are described in detail with reference to FIGS. 6A-6B hereinafter. At step seven (7) neurologic proprioceptive feedback is sent to the object's central nervous system during walking with the LPACT wearable device to train the object for proper balanced movement. The balanced movement affect the object's Neuromuscular system to reset the mechanoreceptors of the Neuromuscular system for future balanced body alignment according to the new proper alignment obtained during walking with the wearable device. Although the object walks with the LPACT wearable device for few minutes a day only, the object's Neuromuscular system is calibrated during the balanced walking session for symmetrical function for long lasting functional balance.

In more details the posture balance training process of LPACT device 100 is obtained as follow: at least two stretch sensors 112 are embedded at the anterior portion of the upper compressive belt 110, attached in proximity to the ASIS anatomical structures 206 on both sides of the pelvis 200. The sensors measure continuously anterior pelvic movement from side to side, during walking or running motion, by comparing pelvic rotation angles α between both sides of the pelvis.

Alternatively, position and/or movement sensors such as Gyro-Accelerometer type, embedded in the upper belt at the mid-anterior or posterior sections may be used as well.

The detection of pelvic rotation angles asymmetry between the right and the left sides of the pelvis when walking is obtained from data collected from the sensors and analyzed by control unit 130. Generally, the sensors from each side of the anterior pelvis ASIS read the anterior pelvic pressure force being applied. The data is being analyzed by the control unit to calculate both sides pelvic rotation angles and the difference between them. Based on that data, a counter pressures command is sent to the posterior elements actuators to apply corrective mechanical pressures to align pelvic motion while walking. In other words, upon detection of asymmetry, the control unit transmits corrective commands, respectively to the actuators at the back of the device. Only the relevant actuator is operated to apply a corrective force in a level and position as determined by the control unit based on real time data analysis.

Distribution of pressures allocation between the four actuators exerts opposing balancing pressure-force to balance between pelvic angles to realign the pelvic motion to be balanced between the right and left sides of the pelvis. The actuators sets-off, coordinate and control variable pressures on the posterior pelvic anatomical structures (i.e., the left and right PSIS and left and right Ischial Tuberosity), which oppose to the asymmetric forces of an unbalanced pelvis while walking. The LPACT wearable device target action is to strive for continuous comparison for balance, in real time, between pelvic rotation angles while walking. An algorithm for distribution of continuous corrective pressures which is based on pelvic motion analysis by the sensors, is applied to correct any mechanical failure of Lumbo-Pelvic posture.

There are six configurations of mechanical failures for imbalanced pelvis that are known in the art. These configurations are prone to correction by the LPACT wearable device of the invention for regaining a balanced posture. Detailed description of some Lumbo-Pelvic positional faulty conditions is provided with reference to FIGS. 6A-6B hereinbelow.

FIG. 5 is a schematic back view illustration of a walking object showing the pelvic rotation angle α created during right gait. The sensors of the LAPACT wearable device provide data about angle α and further about the parallel angle created during left gait. The data is analyzed and the rotation pelvic angle during right gait is compared to the rotation angle during left gait. Asymmetry between the right and left side indicate Lumbo Pelvic posture imbalance.

FIGS. 6A-6B are schematic illustrations of actuating balancing forces in two optional configurations of Lumbo-Pelvic positional faulty conditions. As mentioned above, one main object of the LPACT posture balancing device of the invention is recognition of pelvis rotation asymmetry that can indicate one or more structural failure of the pelvic position.

There are six configurations of mechanical failures for imbalanced pelvis known in the art. These Lumbo-Pelvic positional faulty conditions are defined as Subluxations and describe the anatomical position of each of its components in three-dimensional space. All these locked in space pose conditions are affecting the functional movement of the Lumbo-Pelvic area and as a result create positional asymmetry which may change the Pelvic Angles ratio. The Lumbo-Pelvic

Asymmetrical faulty anatomical posture configurations to be corrected, or rebalanced for symmetry, by the device are set below:
1. Left Posterior-Inferior Ilium/Right Anterior Superior Ilium/Left Sacral base Drop, Left L-5 Vertebral Rotation. (LT-PI/RT-AS/LT. SAC BASE DROP/LT. L5 Rot).
2. Left Anterior-Superior Ilium/Right Posterior-Inferior Ilium/Right Sacral base Drop/Right L-5 Vertebral Rotation. (LT-AS/RT-PI/RT. SAC BASE DROP/RT. L5 Rot.)
3. Right Posterior-Inferior Ilium (RT-PI)
4. Right Anterior-Superior Ilium (RT-AS)
5. Left Posterior-Inferior Ilium (LT-PI)
6. Left Anterior-Superior Ilium (LT-AS)

Configurations number 1-2 are configurations of Lumbo-Pelvic Subluxations that involve faulty positional integrated locked motion of pelvic and Lumbar vertebrae joints. Configurations number 3-6 are Configurations of Pelvic Subluxations that involve faulty positional locked motion of pelvic joints only. The following examples supported by illustrations 6A-6B will demonstrate two configurations of mechanical failures and correction of these failures by the LPACT wearable device of the invention.

### Example 1—Configuration No. 1—LT-PI/RT-AS/LT. SAC BASE DROP/LT. L5 Rot

Configuration No. 1 refers to a positional fault whereas the Left superior portion of the pelvis (Lt. Ilium) is rotated Posteriorly and Inferiorly, whereas the Right superior portion of the pelvis (Rt. Ilium) is rotated in the opposite direction—Anteriorly and Superiorly. In addition, the Left Sacral base is dropping to the Left and the L-5 Vertebrae is posteriorly rotated to the left.

When the Lumbo-Pelvic functional area is locked in that relative position, upon walking the Right Pelvic Angle is greater than the Left Pelvic Angle. This means that the object is in a faulty posture anatomical state. In that subluxated position, the L-PACT device would sense it and induce counter-balancing pressures during walking to train Neuro-Muscular control for symmetrical positional movement. In that case the actuators counter-balancing pressures would induce pressure-force on the Left PSIS and the Right Ischial Tuberosity while walking, during training with the device in order to balance gait by balancing pelvic angles. The actuating balancing forces in configuration No. 1 above is illustrated in FIG. 6A.

### Example 2— Configuration No. 2—RT-AS

This example refers to a positional fault subluxation whereas the Right superior portion of the pelvis (Rt. Ilium) is rotated and locked Anteriorly and Superiorly with no other locked motion at other Lumbo-Pelvic joints. That subluxated position creates a Right greater pelvic angle than the Left. In that case the actuators counter-balancing pressures would induce pressure-force mainly on the Right Ischial Tuberosity while walking during training with the LPACT device as illustrated in FIG. 6B in order to balance gait by balancing the pelvic angles. The Actuators induce counteracting compressive pressure-force to a subluxated locked position in order to direct the pelvis to symmetrical motion while walking. That action limits the motion of hypermobile joints and amplifying the motion of hypomobile joints at the functional area of the pelvis, i.e., at the Lumbo-Pelvic anatomical structure as related to its functional movement. During movement (walking), functional motion of the lumbar vertebrae is dependable on pelvic motion. Any limited, locked or subluxated pelvic motion will affect the position of forces being acted on the lumbar vertebrae. By balancing the pelvis to symmetrical functional movement, the lumbar spine follows the correction and re-aligning too. By the aid of a walking opposing movement to the compressive directive forces which are being applied on to the pelvis, the device directs the pelvis to a corrective symmetrical mechanical motion. That in turn, trains Neuro-Muscular Proprioceptors and Mechano-receptors at the related joints, muscles, ligaments, tendons, and fascia for wiring proper movement control to neurologic pathways. The combined neuro-mechanical training by the aid of walking in restricted balancing pelvic motion, creates long lasting change to spinal aligned posture and for elimination of uneven pressure loads which deteriorate function and cause pain.

FIG. 7 is one example of data view graph of a LPACT system according to embodiments of the invention. The data view graph illustrates the continues Pressure force readings from two sensors positioned at the upper anterior belt 110 of the device. Each of the sensors 112 is located at the ASIS and reads the pressure/stretch force which is being applied by each sides of the pelvis during bilateral motion during walking.

In this specific example, the upper line represents the left side of the pelvis ASIS, whereas the bottom line represents the right side of the pelvis ASIS. The difference between the pelvic anterior pressure applying of both sides is significantly shown, which points the Asymmetrical function of the pelvis. Based on that data, both pelvic rotation angles, the left and the right, are calculated, for applying correlative counter balancing pressure by the posterior actuators to mechanically align the pelvic motion during walking. The goal of the correction is to overlap both lines during a walk as to balance pelvic motion from side to side for a symmetrical pelvic function.

In accordance with embodiments of the invention the LPACT wearable device may be connected to a smart device such as but not limited to smart phone, smart watch, smart bracelet, tablet, fablets, laptop, personal computer. The smart device communicates with the LPACT device by a dedicated software or App and configured to allow real-time monitoring and assessment of the pelvic angle. The LPACT wearable device may be used by therapist and allows the therapist to operate, control and follow the training modules of the system, in a personalized and time-controlled manner, or by the object itself.

In one optional embodiment, the App/software is configured and operable to show the pelvic angels of an object that is wearing the LPACT device by visual representation of the pelvis on an avatar image during gait motion with data of delta differences between angles at both sides of the pelvis in real time and record it. The App/software is configured to perform at least one of the following activities: to ccustomize representation of the Pelvic condition as assessed by the sensors; Data collection and upload to a dedicated cloud server and according to patient affiliation; Uploading patient historic data and comparison tools as to assess the training; Assessing progress of any physical treatment directed to balance pelvic motion even with no relation to the device correction. Additionally, the application preferably allows remote monitoring of pelvic angel by the therapist it should be clear that the above listed activities are only some optional none limiting examples and further activities and parameters may be tracked and operated by the dedicated App/software.

The training of the object's body to balanced symmetric posture is obtained by controlling the actuators function (activate/deactivate pressure, pressure parameters); collection and representation of pressure and time parameters of each actuator; uploading historic training data of each object; and resetting each actuator to basic condition. In some embodiments, the App/software preferably allows a remote manual mode for activation of the actuators by the object or by the therapist. The application may provide statistical data regarding the progression and effectiveness of the treatment to the therapist, the object and the OEM/ODM database.

It should be clear that the description of the embodiments and attached FIGS. set forth in this specification serves only for a better understanding of the invention, without limiting its scope. It should also be clear that a person skilled in the art, after reading the present specification could make adjustments or amendments to the attached Figures and above-described embodiments that would still be covered by the present invention.

1. A wearable device for active neuro mechanical training configured to be worn around a pelvis of an object, comprising:
(a) a first compressive element configured to embrace the ASIS and PSIS anatomical structures of the object pelvis, and to be fastened on these anatomical structures by at least one fastening element;
(b) a second compressive element configure to embrace the Ischial Tuberosities and Greater Trochanter anatomical structures of the object pelvis and to be fastened on these anatomical structures by at least one fastening element;
(c) at least one sensor for sensing pelvic rotation asymmetry of the object in real time during stride motion said at least one sensor is positioned on at least one compressive element;
(d) at least two expandable actuators for applying a corrective pressure on at least one PSIS and/or Ischial Tuberosities in real time during said stride motion so as to balance a measured pelvis rotation asymmetry of the object;
(e) at least two size adjusting elements to allow positioning of said at least one sensor and actuators onto the anatomical structures according to the specific dimensions of the object;
(f) at least two fastening elements for securing said first and second compressive elements onto the anatomical structures of the object pelvis; and
(g) a control unit;
wherein, said control unit is configured and operable to receive data in real time from said at least one sensor during the stride motion, calculate the pelvis symmetry based on the data received, and activate at least one of said expandable actuators to apply an adaptive corrective pressure upon recognition of a pelvis rotation asymmetry.

2. The wearable device according to claim 1, wherein said sensor is at least two sensors positioned on the first compressive element onto the right and left ASIS anatomical elements and configured to provide data about the pelvic rotation angle during right gait and left gait, or at least one sensor configured to be positioned on the vertebrates of the object and to provide data indicative of Pelvis motion along X, Y, Z axis during stride motion, or combination thereof.

3. The wearable device according to claim 1, wherein said sensor is selected from the group consisting of: stretch sensors, motion sensors, pressure sensors, location sensors, electronic skin sensors, and conductive sensors.

4. The wearable device according to claim 1, wherein said at least two expandable actuators are positioned in a manner that at least one actuator is placed on the first compressive element onto the right and/or left PSIS anatomical elements, and at least one another actuator is placed on the second compressive element onto the right and/or left Ischial Tuberosities.

5. The wearable device according to claim 1, wherein said at least one sensor is continuously sending data to said control unit during stride motion and the correction of the pelvis rotation asymmetry detected is performed in real time by adjusting the corrective pressure level applied by said actuators until the right and left rotation angles of the pelvis are similar.

6. The wearable device according to claim 1, wherein the stride motion and pelvis asymmetry detection and correction are performed in periodic training sessions of up to ten minutes daily, weekly or monthly according to the pelvis rotation asymmetry detected.

7. The wearable device according to claim 1, wherein said expandable actuators are volume-controlled elements and the pressure imposed to balance the pelvis asymmetry is correlative to the volume of the actuator in use.

8. The wearable device according to claim 1, wherein said expandable actuators are either one of the following elements: a) inflatable elements configured to be inflated with gas; b) electroactive elements (EAP's) configured to change volume upon changing physical state; and c) fabric soft pneumatic actuators (FSPAs).

9. The wearable device according to claim 8 wherein said electroactive elements are made of electroactive polymers selected from: dielectric elastomers, electro responsive polymers, Ionic electroactive polymers (IEAP's) and conductive polymers.

10. The wearable device according to claim 1, wherein said expandable actuators are capable to change volume in a differential manner such that the pressure applied in one area of the actuator is different than the pressure applied in another area of the actuator.

11. The wearable device according to claim 1, wherein said first compressive element and said second compressive element are housed together in a form of any one of the following: a belt, a garment, and a pouch.

12. The wearable device according to claim 1, further comprising a screen.

13. The wearable device according to claim 1, wherein said at least two size adjusting elements are configured to allow positioning of said at least one sensor and actuators onto the anatomical structures are at least one slider connected to a sliding strip to thereby allow sliding of at least one of the compressive elements upward and downward on the sliding strip for positioning said actuators and sensors on said anatomical structures of the pelvis according to the specific dimensions of the object.

14. The wearable device according to claim 1 further comprising wireless communication elements to allow connection of the wearable device to a smart device, said smart device comprises a supportive software or App for communicating with the wearable device.

15. The wearable device according to claim 14, wherein the analysis of the data received from the at least one sensor, determining pelvis asymmetry and calculation of the corrective force to be applied by the expandable actuators on the object's pelvis to reach symmetry are calculated in real time by the control unit or by the smart device or by a remote computer.

16. The wearable device according to claim 1, wherein a combination of forces is applied on the object's pelvis by the device for correction of the asymmetry detected between the right side and the left side of the pelvis during stride, said forces are combined of pre-tensed pressure of the compressive elements, the corrective force applied on the specific anatomical structures by the expandable actuators, and the stride motion itself.

17. The wearable device according to claim 1, wherein said first compressive element and second compressive element apply pretension of the object's pelvis.

18. A Lumbo Pelvic active correction training device for reduction and prevention of back pain, said balancing device is a wearable device configured to allow sensing bilateral pelvic angle of an object, comparing the right and left pelvic rotation angles and correcting a pelvic asymmetry according to the data measured in real time during stride motion, and wherein said Lumbo Pelvic active correction training device comprising a first compressive element configured to embrace the ASIS and PSIS anatomical structures of the object pelvis; a second compressive element configure to embrace the Ischial Tuberosities and Greater Trochanter anatomical structures of the object pelvis; at least one sensor for sensing pelvic rotation asymmetry of the object in real time during the stride motion, said at least one sensor is positioned on at least one compressive element at least two expandable actuators for applying a corrective pressure on at least one PSIS and/or Ischial Tuberosities in real time during said stride motion so as to balance a measured pelvis rotation asymmetry of the object at least two size adjusting elements to allow positioning of said at least one sensor and actuators onto the said anatomical structures according to the specific dimensions of the object and a control unit.

19. (canceled)

20. The Lumbo Pelvic active correction training device according to claim 18 wherein said at least one sensor is at least two sensors positioned on the first compressive element onto the right and left ASIS anatomical elements and configured to provide data about the pelvic rotation angle during right gait and left gait, or said at least one sensor configured to be positioned on the vertebrates of the object and to provide data indicative of Pelvis motion along X, Y, Z axis during stride motion, or combination thereof.

21. The Lumbo Pelvic active correction training device according to claim 18, wherein said at least two expandable actuators are positioned in a manner that at least one actuator is placed on the first compressive element onto the right and/or left PSIS anatomical elements, and at least one another actuator is placed on the second compressive element onto the right and/or left Ischial Tuberosities.

22. The Lumbo Pelvic active correction training device according to claim 18, wherein said at least one sensor sensor is continuously sending data to said control unit during stride motion and the correction of the pelvis rotation asymmetry detected is performed by adjusting the corrective pressure level applied by said actuators in real time until the right and left rotation angles of the pelvis are similar.

23. The Lumbo Pelvic active correction training device according to claim 18, wherein the stride motion and pelvis asymmetry detection and correction are performed in periodic training sessions of up to ten minutes daily, weekly or monthly according to the pelvis rotation asymmetry detected.

24. The Lumbo Pelvic active correction training device according to claim 18, wherein said expandable actuators are volume-controlled elements and the pressure imposed to balance the pelvis asymmetry is correlative to the volume of the actuator in use.

25. The Lumbo Pelvic active correction training device according to claim 18, wherein said expandable actuators are either one of the following elements: a) inflatable elements configured to be inflated with gas; b) electroactive elements (EAP's) configured to change volume upon changing physical state; and c) fabric soft pneumatic actuators (FSPAs).

26. The Lumbo Pelvic active correction training device according to claim 18, wherein said expandable actuators are capable to change volume in a differential manner such that the pressure applied in one area of the actuator is different than the pressure applied in another area of the actuator.

27. The Lumbo Pelvic active correction training device according to claim 18, wherein said first compressive element and said second compressive element are housed together in a form of any one of the following: a belt, a garment, and a pouch.

28. The Lumbo Pelvic active correction training device according claim 18 further comprising a screen.

29. The Lumbo Pelvic active correction training device according to claim 18 wherein said at least two size adjusting element to allow positioning of said at least one sensor and actuators onto the anatomical structures are at least one slider connected to a sliding strip to thereby allow sliding of at least one of the compressive elements upward and downward on the sliding strip for positioning said actuators and at least one sensor on said anatomical structures of the pelvis according to the specific dimensions of the object.

30. The Lumbo Pelvic active correction training device according to claim 18 further comprising a wireless communication element so as to allow connection of the device to a smart device, said smart device comprises a supportive software or App for communicating with the device.

31. The Lumbo Pelvic active correction training device according to claim 18, wherein the analysis of the data received from the sensors, determining pelvis asymmetry and calculation of the corrective force to be applied on the object pelvis to reach symmetry are calculated in real time by the control unit or by the smart device or by a remote computer.

32. The Lumbo Pelvic active correction training device according to claim 18, wherein a combination of forces is applied on the object's pelvis by the device for correction of the asymmetry detected between the right side and the left side of the pelvis during stride, said forces are combined of pre-tensed pressure of the compressive elements, the corrective force applied on the specific anatomical structures by the expandable actuators, and the stride motion itself.

33. The Lumbo Pelvic active correction training device according to claim 18, wherein said first compressive element and second compressive element apply pretension of the object's pelvis.

34. (canceled)

35. (canceled)

36. (canceled)

37. (canceled)

38. (canceled)

39. (canceled)
